(19) United States
(12) Patent Application Publication
(10) Pub. No.: US 2016/0022753 A1
(43) Pub. Date: Jan. 28, 2016

(54) COMPOSITIONS DERIVED FROM SWEET POTATO GREENS AND METHODS OF PREPARATION AND USE

(71) Applicant: GEORGIA STATE UNIVERSITY RESEARCH FOUNDATION, INC., Atlanta, GA (US)

(72) Inventors: Ritu Aneja, Liburn, GA (US); Sushma Reddy Gundala, Atlanta, GA (US)

(21) Appl. No.: 14/777,116

(22) PCT Filed: Mar. 15, 2014
(86) PCT No.: PCT/US14/30026

§ 371 Date: Sep. 15, 2015

Related U.S. Application Data
(60) Provisional application No. 61/787,251, filed on Mar. 15, 2013.

(51) Int. Cl. A61K 36/39; G01N 33/50

(52) U.S. Cl. CPC A61K 36/39; G01N 33/5011

(57) ABSTRACT

The present invention includes methods of identifying a fraction of a sweet potato greens extract (SPGE) that is useful in reducing the risk of or treating, cancer, hypertension, diabetes, or a wound; compositions including the identified fraction(s); methods of administering SPGEs or fractions thereof; and uses of the compositions described herein in the preparation of a medicament.

### CROSS-REFERENCE TO RELATED APPLICATIONS

This application claims the benefit of the filing date of U.S. Application No. 61/787,251, which was filed Mar. 15, 2013.

### FIELD OF THE INVENTION

The present invention features compositions, including physiologically acceptable formulations intended for oral administration, that are obtained from the greens of sweet potatoes. The compositions are useful as dietary supplements and/or therapeutic agents. More specifically, our studies to date indicate that certain fractions of sweet potato greens have beneficial (e.g., chemopreventive) properties and are capable of, for example, inhibiting the growth of tumor cells and/or inducing apoptosis.

### BACKGROUND

Nutrition research has long favored a reductionist approach that emphasizes the health benefits of single phytochemicals. However, the notion that constituent phytochemicals present in whole foods may act in synergy is gaining momentum. Recent studies suggest that whole or partially purified extracts of a plant offer significant advantages over single, isolated ingredients. For example, such extracts may have stronger anti-proliferative activity when applied to human colon and hepatic cancer cells, and they may provide for improved absorption, metabolism, or retention of the bioactive food components. Recent data support the idea that the health benefits achieved from consuming fruits and vegetables may not result solely from isolated single compounds, but rather that these benefits arise due to additive and/or synergistic interactions among components that “partner” with one another.

### SUMMARY

The present invention is based on our work with extracts of sweet potato greens (SPGE). We have fractionated the complex, whole extract and sequentially separated it further into sub-fractions based upon physicochemical characteristics such as polarity and solubility. More specifically, the compositions and methods described herein relate, at least in part, to our studies demonstrating that a polar fraction, F5, exhibits significant anti-proliferative activity in prostate cancer cells both in vitro and in vivo.

The compositions of the present invention encompass physiologically acceptable (e.g., non-toxic) compositions that include effective amounts of major phenolics, including quinic acid (QA), chlorogenic acid (ChA), and caffeic acid (CA). The present compositions, methods, and uses can be employed to promote wellness in healthy individuals by reducing the risk of cancer or another condition described herein (e.g., hypertension or diabetes). A “healthy” individual is a subject (including, but not limited to, a human being) that does not exhibit the signs and symptoms of a condition for which the present compositions are indicated or administered. For example, the present compositions can be administered to reduce the risk of prostate cancer in a human male who has not been diagnosed as having prostate cancer; to reduce the risk of hypertension in a human whose blood pressure is still within normal limits but perhaps at heightened risk for hypertension due to family history; or to reduce the risk of diabetes in a human who metabolizes glucose normally. These subjects may have some other ailment, but they are healthy in that they have not been diagnosed as having cancer, hypertension, or diabetes, respectively; a subject need not be healthy in every respect. In addition to reducing risk, the present compositions, methods, and uses can be administered to treat a patient who has developed cancer, hypertension, or diabetes or who has sustained a wound.

Accordingly, in a first aspect, the invention features methods of identifying a useful fraction of a sweet potato greens extract (SPGE), such as the F5 fraction obtained as described herein. The methods can be carried out by a series of steps that include: (a) providing a SPGE; (b) fractionating the SPGE; and (c) identifying the fraction or fractions that have (i) an improved ability to inhibit the development, proliferation, and/or metastasis of cancer cells relative to an unfractionated SPGE and (ii) a higher concentration of quinic acid and chlorogenic acid than an unfractionated SPGE. Alternatively, in step (c)(i), one can assay for an improved ability to inhibit the development of, or to reduce, hypertension; to inhibit the development of, or to treat, diabetes; or to promote wound healing. The methods may include the further step of: (d) isolating the fraction or fractions identified in step (c) and, optionally, formulating the fraction or fractions in unit dosage form for administration (e.g., oral or topical administration). The ability to inhibit the proliferation of cancer cells can be tested by the cell culture and in vivo assays described herein and/or by cell growth, proliferation, and apoptosis assays known in the art. Similarly, one can employ cell culture or animal models of hypertension, diabetes, and wound healing. SPGEs from a number of different varieties of sweet potatoes, including the sweet potato Ipomoea batatas, can be used, with the expectation that there will be variations in the amounts of major phenolics among varieties (e.g., when the extract and fractions are obtained from Jewel potatoes or TU155). The methods of the invention (e.g., making an SPGE and/or identifying a useful fraction therein) can be carried out with any variety of sweet potato, even though the resulting composition may vary with factors such as the age of the greens at the time of harvest and the mode of processing the leaves. For example, the methods can be carried out with leaves harvested after 30, 45, 60, or 75 days of growth and with leaves that are air-dried, frozen or freeze-dried). Other conditions that may influence the precise content of the extracts or a fraction thereof include the cultivation season, soil properties, and amount of rainfall. More specifically, in the present methods, the SPGE can be prepared by soaking air-dried sweet potato leaves in an alcohol (e.g., methanol) for about three consecutive days; collecting the supernatant; concentrating the supernatant (e.g., in vacuo); and drying (e.g., freeze-drying) the supernatant to a solid-powder form. Such forms are within the scope of the present invention. Fractionating the SPGE can be carried out by any method known in the art, including methods in which the SPGE is passed over a column (e.g., a silica gel column) and fractions of the SPGE are eluted from the column. To obtain the fractions, the column can be eluted using, for example, an alkane, mixtures of the alkane and ethyl acetate, mixtures of the ethyl acetate and an alcohol, and the alcohol. For example, one can elute the column with: (a) 100% hexane; (b) a hexane- and ethyl acetate-containing solution, wherein the ratio of hexane to ethyl acetate changes from primarily hexane to primarily ethyl acetate over the course of subsequent elutions; (c) an ethyl acetate- and methanol-containing solution, wherein the ratio of methanol to ethyl acetate changes from primarily ethyl acetate to primarily methanol over the course of subsequent elutions; and (d) 100% methanol. In step (b), where the ratio of hexane to ethyl acetate changes, one can begin with a mixture of hexane:ethyl acetate in a ratio of about 90:10, then progress to elutions at ratios of 80:20, 70:30, 60:40, and 50:50. This can be followed with 100% ethyl acetate. Similarly, in step (c), where the ratio of methanol:ethyl acetate changes with subsequent elutions, one can use mixtures of methanol:ethyl acetate of 0:90, then 20:80, then 30:70, 40:60, 50:50, and then 60:40, 70:30, 80:20, and 90:10. Elution with 100% methanol can follow.

Fractionating the SPGE can include various chromatographic methods, including column chromatography and thin layer chromatography (TLC). For example, the fractions obtained following column chromatography can be concentrated in vacuo and then characterized by TLC. Fractions with similar TLC profiles (R_{f} values) can be pooled. Using such methods, one can obtain about 5-10 fractions, one or more of which will demonstrate (i) an improved ability to inhibit the proliferation of cancer cells (e.g., prostate cancer cells) relative to an unfractionated SPGE and (ii) a composition with a higher concentration of QA and ChA than an unfractionated SPGE. In other embodiments, the fraction(s) may also demonstrate, or alternatively demonstrate a positive effect in the management of hypertension, diabetes, and/or wound healing. The desired fraction(s) can also include neochlorogenic acid, cryptochlorogenic acid, quercetin-glucoside, quercetin, astragalin, or a combination thereof. As noted, SPGE can be fractionated by methods other than the one described above. For example, fractionation can be achieved by extraction with carbon dioxide or a supercritical fluid or by distillation with water. CO₂ extraction and supercritical fluid extraction (SFE) are known as effective ways of extracting beneficial essences from plant matter because their high diffusion rates allow faster penetration of solids than a liquid solvent. Also, CO₂ does not leave residues behind. The invention encompasses physiologically or pharmaceutically acceptable formulations that include a fraction of SPGE having characteristics as described herein, regardless of the method by which the formulation was obtained.

In another aspect, the invention features physiologically acceptable formulations that include a fraction of SPGE that includes quinic acid (QA) and chlorogenic acid (ChA) in amounts or in a ratio relative to one another that is different from that found in a comparable but unfractionated SPGE. The fraction may also include caffeic acid in amounts that are lower than those found in the unfractionated SPGE. Thus, useful formulations can include fractions having elevated levels of quinic acid and/or chlorogenic acid relative to the levels found in a comparable but unfractionated SPGE. For example, the fraction can include at least or about 2.5 times the amount of quinic acid as in the comparable but unfractionated SPGE and/or at least or about 2.5 times the amount of chlorogenic acid in the comparable, unfractionated SPGE. With respect to these three agents, the ratio of QA:ChA:CA can be about 6 to 1 to 0.007, and the ratio of ChA to CA can be less than about 1:1. By “about” we mean within 25% of the value provided. For example, about 2.5 times the amount of quinic acid can range from 2.25-2.75 times the amount of quinic acid. Any of the formulations can also include one or more of neochlorogenic acid, cryptochloro-genic acid, quercetin-glucoside, quercetin, astragalin, or combinations thereof.

For administration to a patient, any of the formulations can also include an inhibitor of uridine 5′-diphospho-glucuronosyltransferase (UDP-glucuronosyltransferase, UGT), an enzyme within the class glycosyltransferase that catalyzes the transfer of the glucuronic acid component of UDP-glucuronic acid to a small hydrophobic molecule. Useful inhibitors include eugenol, piperine, and curcumin. The UGT targeted can be UGT1A1 (which can be inhibited with atazanavir, gemfibrozil, indinavir, or ketoconazole), UGT1A3 (which can be inhibited by gemfibrozil), UGT1A4, UGT1A6, UGT1A8, UGT1A9, UGT1A10, UGT2B4, UGT2B7 (which can be inhibited by ketoconazole or valproic acid) or UGT2B15. UGT1A1, UGT1A6, UGT1A9, UGT2B7, and UGT2B15 can also be inhibited by the herbals Silybum marianum and Valeriana officinalis. One can use these ingredients together with a SPGE or one or more fractions thereof in the preparation of a medicament (e.g., a medicament for reducing the risk of, or treating, cancer, hypertension, diabetes, or a wound). Similarly, the methods of the invention encompass administration of an inhibitor of UGT together with an SPGE or one or more fractions thereof. The inhibitor of UGT and the SPGE or the fraction thereof can be administered by the same or distinct routes of administration and administered either simultaneously or sequentially.

In addition, the present compositions can include an excipient, such as a filler, hydrogel, buffer, coloring agent, or flavoring agent. For convenience, the formulations can include unit dosages suitable for oral administration, but the invention is not so limited (other formulations and routes of administration can be made and used as well). When formulated and taken as dietary supplements, the present compositions can be administered routinely (e.g., daily) and prophylactically; when formulated and prescribed as therapeutic agents, they can be administered in the event of cancer (e.g., to a patient suffering from prostate cancer).

In the description that follows, we describe a bioactivity-guided separation of SPGE that produced a polyphenol-enriched fraction (F5) including the major phenols quinic acid (QA), chlorogenic acid (ChA) and (in lesser amounts) caffeic acid (CA), as well as isochlorogenic acids, 4,5-di-CQA, 3,5-di-CQA and 3,4-di-CQA in a distinct composition. This work supports the present methods of identifying and/or isolating a fraction of SPGE (e.g., F5 or a fraction with comparable characteristics) and our claims to the fractionated material produced by such methods. The invention encompasses the material described as F5 or “fraction 5” and dosage forms containing this material (e.g., a dosage form suitable for administration (e.g., oral or intravenous administration) to a human). Regardless of the method by which the compositions are produced (e.g., whether by separation techniques such as those described herein or by synthetic or combinatorial methods), the invention features compositions comprising certain amounts of QA and ChA or certain amounts of QA, ChA, and CA, including the amounts described herein. For example, the compositions can include QA, ChA and CA in a ratio relative to one another that delays the onset of cancer and/or retards the progression of cancer.

Other advantages, features, and embodiments of the present compositions and methods are described in the drawings, description, and the claims below.

### BRIEF DESCRIPTION OF THE DRAWINGS

FIG. 1 is a bar graph showing the concentration (mg/L) of ChA equivalents for seven fractions of SPGE (F1-F7) and for the SPGE itself. The phenolic content was determined as described in Example 1 via the Folic-Ciocalteu method.

FIGS. 2A and 2B are line graphs showing the % of surviving PC-3 cells at various concentrations (0.001-100 μg/ml) of the indicated fractions of SPGE. In FIG. 2A, seven fractions were tested. In FIG. 2B, fraction F5 was tested further, as described in Example 2.

FIG. 3 is a series of graphs relevant to the experiments described in Example 4. In panel 3Ai, a chromatograph shows the relative abundance of the major phenolics and isochlorogenic agents in the SPGE, which were divided between fractions F5-A and F5-B (panel 3Aii). The results obtained when fractions F5, F5-A, and F5B were tested in a cell survival assay are shown in panel 3B. Individual phenolics and a combination thereof (QA+ChA+CA) were also tested for their ability to inhibit cell growth (panel 3C).

FIGS. 4A and 4B are line graphs illustrating data as described in Example 6, obtained from an in vivo model of prostate cancer in nude mice. As shown in panel 4B, total tumor volume was found to be greatly reduced in F5-treated mice relative to control.

### DETAILED DESCRIPTION

Polyphenols are well known for their abundance in fruits and vegetables, and they are known to provide anticancer benefits upon regular consumption. They are versatile molecules containing several hydroxyl groups with multiple aromatic rings (the structures of which are readily available in the art). The amphiphilic phenolic moiety of polyphenols blends the hydrophobic character of its planar aromatic core with the hydrophilic nature of its polar hydroxy substituent (Quideau et al., “Plant Polyphenols: Chemical Properties, Biological Activities, and Synthesis” Angew Chem Int Ed Engl., 2011). The inherent bio-physicochemical properties of the phenolic group display a variety of functional roles, including plant resistance against microbial pathogens, and protection against solar radiation. Epidemiological studies have linked the consumption of polyphenol-rich foods like cocoa, red wine, tea, fruits, vegetables, etc. with a lower incidence of chronic diseases including cancer (Yang et al., Annu. Rev. Nutr. 21:381-406, 2001). Although it is easy to evaluate the protective effect of a single phytochemical (e.g., a single polyphenolic compound), the health benefits of dietary polyphenols are difficult to discern when numerous phytochemicals, including polyphenolics, flavonoids, lignans and tannins are active and working synergistically. The complexity of polyphenols in foods limits the identification of definitive compositions of partially purified extracts that display superior efficacy compared to single-agents or whole foods. Fractionation, however, of a whole extract may result in the increased concentration of bioactive constituents in a particular sub-fraction, thus enhancing efficacy.

Sweet potato greens (SPG), for example, from Ipomoea batatas, are commercially available and a significant source of dietary polyphenols. SPG are also widely consumed as a fresh vegetable in Asia, in particular, Taiwan and China. Caffeic, monocaffeoylquinic (chlorogenic acid), dicaffeoylquinic and tricaffeoylquinic acids are major phenolic constituents of SPG (Mosha and Gaga, Plant Foods Hum. Nutr. 54:271-83, 1999; Kurata et al., J. Agric. Food Chem. 55:185-90, 2007). SPG have been shown to have radical-scavenging, anti-mutagenic, antidiabetic, antibacterial, anti-inflammatory, and anticancer activities (Islam, J. Food Sci. 71:R13-R121, 2006; Huang et al., Int. J. Food Sci. Nutr. X:1-9, 2007). Extracts of SPG (SPGE) are non-toxic and inhibit prostate growth in vitro and in vivo (Karna et al., Carinogenesis 32:1872-80, 2011). Recently, we demonstrated the growth-inhibitory and apotosis-inducing properties of polyphenol-rich sweet potato greens extract (SPGE) in cell culture and in vivo prostate cancer xenograft models. These results, which are presented in the Examples below, emphasize the importance of synergistic interactions among various bioactive components to confer remarkable in vitro and in vivo effects in prostate cancer models.

The composition of whole SPGEs and sub-fractions thereof may be dependent on the variety of the potato (e.g., Jewel or TU155), the age of the leaves at the time of harvest (e.g., they may be between 20-100 days (e.g., harvested around day 30, day 45, day 60 or day 75), and the mode of processing of the leaves (e.g., by air drying, freezing, or freeze-drying, all of which can be used in the present methods). Several other variables such as the cultivation season, soil, amount of rainfall, etc. are also likely to influence the nature and composition of the extract. These varietal differences in total phenolic content, in the content and relative amounts of QA, ChA, CA, and isochlorogenic acid content, among other unknown components, may possibly affect the antiproliferative efficacy of the whole extract and its derived fractions, thus making the known characteristics of the derived fractions all the more important.

One of the bioactive constituents found in SPGE, QA is enriched in F5 compared to the parent extract, and fractions prepared and used in the context of the invention can be enriched in QA to about the same extent. “About” or “approximately” as used herein with respect to any characteristic generally means within 25% (e.g., within 5-10%, inclusive) of a given value or range. Moreover, the numerical quantities given herein are approximate, meaning that the term “about” or “approximately” can be inferred if not expressly stated. It has been reported that quinic acid is not responsible for any known efficacy, but it may play a nutritionally supportive role. For example, studies have shown that quinic acid supports the in situ synthesis of essential metabolites like tryptophan and nicotinamide in the gastrointestinal tract. This, in turn, leads to DNA repair enhancement and NF-kB inhibition via increased nicotinamide and tryptophan production (Pero et al., Phytother. Res. 23:335-46, 2009). The other phenolic acids in F5, namely CA and ChA, belong to an abundant class of polyphenols called hydroxycinnamic acids, which are widely present in a large variety of fruits and vegetables. Caffeic acid (CA) is the major representative of this class, and it exists extensively as a conjugate with quinic acid (QA) as seen in chlorogenic acid. It is well known that the bioavailability and efficacy of these hydroxycinnamic acids depend on their uptake and metabolism in the gut mucosa (Manach, et al., Am. J. Clin. Nutr. 79:727-47, 2004). While CA is readily absorbed in the small intestine and detected in the blood, ChA is poorly absorbed and is detected only in urine unchanged, indicating the differential metabolism of these compounds. The metabolism of ChA is not well studied and is controversial, as some groups believe that it is usually hydrolyzed into CA and O-methylated metabolites in the lower intestine due to enzymatic reactions by the gut microflora (Lafay et al., Br. J. Nutr. 96:39-46, 2006). This suggests that the bioactivity could be due to CA.

We have carried out a bioactivity-guided fractionation of SPGE based upon differential solvent polarity using chromatographic techniques that led to the identification of the remarkably active, polyphenol-enriched fraction, F5. F5 is about 100-fold more potent than the parent extract, and fractions within the scope of the present invention (whether obtained using the chromatograhic techniques described in the Examples below or other known techniques) can similarly be about 100-fold more potent than the parent extract from which they were derived. Potency can be assessed with respect to any given characteristic, including the ability to perform in an in vitro or in vivo model of cancer (e.g., prostate cancer). Further, and although the fractionation of SPGE using Ipomoea batatas is described in the Examples, the invention is not limited to any particular species of SGPE. Other varieties of SPG that may be used include, but are not limited to those known commonly as Evangeline Sweet Potato™ (see U.S. Plant patent 19710), Bonita (see LaBonte et al., U.S. Plant patent pending), Murasaki-29 (see U.S. Plant patent 19955), Beauregard B-63 (see, HortScience 27:377, 1987), Beauregard B-14 (believed to be similar to Beauregard B-63), O-Henry (also believed to be similar to the Beauregard varieties), Bienville (see, HortScience 38:473-474, 2003), Hernandez (see, HortScience 27:377, 1992), Heartogold (see, LA. AES Annu. Rept., 1947-1948), Porto Rico (PR-6; see, NC AES Bull. 429, 1966), Texas Porto Rico (TX PR; believed to be similar to Porto Rico), Jewel (see, NC Unnumbered Mimeo. Rept. January 1970), LA 07-146 (see LaBonte et al., U.S. Plant patent pending), and Orleans (see LaBonte et al., U.S. Plant patent pending).

As described further below, we tested the efficacy of F5 in preventing disease recurrence as well as imparting chemopreventive benefits. We used a straightforward and holistic approach to make SPGE and have followed a bioactivity-guided fractionation of the whole leaf extract to obtain and identify active fractions/constituents. Generally, in the methods of the invention, any fraction obtained can be tested for bioactivity and a certain content or characteristic as described herein. Fraction 5 (eluted via a 50:50 and 40:60 ethyl acetate and methanol system) is a medium-polar fraction that was identified using mobile-phase systems of varying polarity to elute SPGE down a classical silica gel column to fractionate and elute components of different polarities in different solvent systems that exhibited the highest antiproliferative activity in prostate cancer cells. Since F5 was determined to be ˜100 fold more potent compared to the parent, SPGE, we further investigated F5's composition. Among the repertoire of bioactive polar phenols enriched in F5, we have identified quinic, chlorogenic and caffeic acids in a distinct ratio, which is applicable to fractions obtained from other vegetative sources using other separation techniques. The analytical data revealed higher abundance of QA and ChA over CA in F5 compared to the whole extract. The pattern of QA:ChA:CA in F5 is similar to that in SPGE, but these compounds are highly enriched in F5 compared to the parent whole extract. On the contrary, the signature of isochlorogenic acids in SPGE differs from F5, wherein the most abundant in the whole extract is 3,5-di-CQA and 3,4-di-CQA is maximally present in F5. Accordingly, fractions made and used within the scope of the invention can also be enriched for 3,4-di-CQA. A seven-day stability study suggested a stable shelf-life of F5 when stored at 4° C., as there were no variations in the concentrations of individual constituents that make up this most-active fraction.

The in vitro efficacy of F5 was supported by the study of synergy we performed with the pure standards in combinations. This strengthened the concept that QA, ChA, CA, 4,5-di-CQA, 3,5-di-CQA and 3,4-di-CQA act synergistically among themselves and with other unknown components to exert maximum efficacy, and emphasized the importance of the ratio of phytochemicals for the observed antiproliferative activity. This observation is further supported by the quintessential green tea polyphenol concoction, Polyphenon E, which has been proven to confer optimal anticancer benefits via a specific combination of five different catechins, including epicatechin, gallocatechin gallate, epigallocatechin, epicatechingallate, and most abundantly, EGCG (Bode and Dong, Cancer Prev. Res. 2:514-17, 2009). This specific formulation of green tea is in clinical cancer trials funded by the National Cancer Institute.

We also found that sub-fractions of F5 did not outperform F5, suggesting a potential additive or synergistic interaction among F5 phytochemicals. A similar observation was made by Liu et al. in that sub-fractions of black raspberry extract's active fraction, WBR-95, showed diminished antiangiogenic efficacy compared to the refined parent (Liu et al., J. Agric. Food Chem. 53:3909-15, 2005).

The efficacy of SPGs and any extract or fraction obtained therefrom can be evaluated using any one of several in vivo models known in the art (some of which are described in the Examples below). For instance, to study chemoprevention of adenocarcinomas by SPGs, the transgenic APC min mice model for colon cancer may be used; to study prostate cancer, the transgenic adenocarcinoma of mouse prostate (TRAMP) model can be used. Briefly, the TRAMP model relies on transgenic male mice (inbred C57BL/6) in which the progressive stages of prostate cancer can develop spontaneously over a period of time (the rat probasin promoter induces SV40 T antigen expression in prostate epithelium). The progression observed in this model ranges from mild intraepithelial hyperplasia to large multi-nodular malignant neoplasias. The model is considered the gold standard for studying the four stages of human prostate cancer (initiation, progression, angiogenesis and metastasis). In this study, the female transgenic mice (C57BL/6 strain) will be cross-bred with the non-transgenic males as the resultant transgenic mice will develop prostate tumors. The transgenic incorporation will be determined by PCR-genotyping of tail DNA. The males found positive for Tag transgene will only be used in the study. SPGE/F5 can be administered to the animals by oral-gavage route, starting at 4, 12, 20 or 30 weeks of age (at the doses based on in vitro results). The treatment groups will then be sacrificed at 12, 20, 30 and 45 weeks along with a control group orally-fed with vehicle matching the age of each treatment group. Once the animals are euthanized, the weight differences in the genitourinary tracts of all the groups will be measured as well as the excised prostate tissues will be identified via immunohistochemical analysis.

Our results suggest that the use of F5 as a non-toxic dietary supplement with specific phytochemicals in defined ratios may be beneficial to bypass the potential limitations in absorption and assimilation of active whole food components, like variations in human genetic profiles affecting nutritional absorption (German, J Am Diet Assoc, 105:530-1). It is also highly likely that the variability of unidentified components in F5 and their standardization to constitute a therapeutic blend may provide valuable insights for a clinical dietary intervention.

Methods of Identifying a Fraction of SPGE:

The methods of the present invention include preparing an extract, determining phenolic content, and fractionation processes. The SPGE extract may be prepared using air-dried, frozen or freeze-dried leaves prior to fractionation. The total phenolic content may be determined by Folin-Ciocalteu method using chlorogenic acid as the standard. SPGE fractionation may be carried out using classical column chromatography, CO₂ extraction or supercritical fluid extraction. The methods of the present invention may also include other steps and methods known in the art.

As noted above, sweet potato greens that can be used in the present methods include, but are not limited to: Ipomoea batatas, Evangeline Sweet Potato™, Bonita, Murasaki-29, Beauregard B-63, Beauregard B-14, O-Henry, Bienville, Hernandez, Heartogold, Porto Rico (PR-6), Texas Porto Rico (TX PR), Jewel, LA 07-146, and Orleans.

Preferred polyphenolic phytochemicals that may be subjected to fractionation methods and further measured or otherwise characterized (e.g., for physical traits such as polarity and for biological traits such as anti-proliferative activity) include but are not limited to: quinic acid, caffeic acid and its ester, chlorogenic acid, and isochlorogenic acids, 4,5-di-CQA, 3,5-di-CQA and 3,4-di-CQA. Other bioactive components that may also be extracted and content determined include but are not limited to: neochlorogenic acid, cryptochlorogenic acid, quercetin-glucoside and its isomer (QnG isomer), quercetin (Qn) and astragalin.

Uses and Conditions:

SPGEs are useful in treating, preventing, reducing the risk of, and/or managing a variety of conditions. While the invention is not limited to compositions, methods, or uses that exert an effect by any particular mechanism, we believe a variety of subjects can benefit from the present invention due to antimicrobial (e.g., anti-bacterial), antidiabetic, antioxidant, and immune system or immune response promoting activities of the SPGEs. Patients who may benefit from treatment with an SPGE or a fraction thereof (e.g., F5) include, without limitation, patients at elevated risk for, or who are suffering from, hypertension, diabetes, a wound (e.g., to the skin and/or underlying organs or tissues) and cancers (e.g., prostate cancer).

Pharmaceutical and Nutraceutical Formulations (Nutritional Supplements), Doses, and Administration:

Pharmaceutical compositions for use as described herein may be formulated in a conventional manner using one or more pharmaceutically or physiologically acceptable carriers or excipients. Varying concentrations of a fraction or fractions of SPGE, F5 per se, an equivalent fraction, or reconstituted bioactive constituents thereof may be used, with the agent(s) being administered in an amount effective to achieve their intended purpose. It is within the ability of one of ordinary skill in the art to determine therapeutically effective or nutritionally effective amounts and formulations for their delivery to a patient. For convenience, oral formulations are preferred.

The specific, therapeutically effective dose level for any particular patient will depend upon a variety of factors including the amount and activity of the F5 or other bioactive constituents; the specific composition and/or formulation used; the age, body weight, general health, sex and diet of the patient; the time of administration, route of administration, and rate of excretion of F5 or other bioactive constituents; the duration of the treatment; drugs used in combination or coincidentally with F5 or other bioactive constituents; and like factors well known in the medical and neutraceutical arts.

The therapeutically effective dose of F5 or other bioactive constituents can be administered using any medically acceptable mode of administration. Moreover, in the event of a combination therapy, F5 or other bioactive constituent can be administered with a second agent in a single dosage form or otherwise administered in combination (e.g., by sequential administration through the same or a different route of administration). Although one would contemplate any of the modes of administration known in the art, preferably the pharmacologic agent is administered according to the recommended mode of administration, for example, the mode of administration listed on the package insert of a commercially available agent. In general, the dose may be contained in a single dose or in divided doses per day, and the absolute amounts can be informed by animal studies, including those we conducted and described below. The concentration ranges of a fraction of SPGE can be calculated by one of ordinary skill in the art. For instance, when the fraction comprises quinic acid, chlorogenic acid and caffeic acid, the ratio of QA to ChA to CA may range from about 6 (e.g., about 4-10) to about 1 to about 0.01 (e.g., about 0.005 to about 0.02). We found the ratiometric relationship between QA, ChA, and CA to be similar in fractions F5 (where it was 6 to 1 to 0.005), F6 (where it was 8.8 to 1 to 0.002), and F7 (where it was 4 to 1 to 0.02). In other fractions encompassed by the invention, these ratios may be about the same as the ratios we found for F5, F6, and/or F7.

The compounds described herein may be administered directly or they may be formulated to include at least one pharmaceutically acceptable carrier, diluent, excipient, adjuvant, filler, buffer, preservative, lubricant, solubilizer, surfactant, wetting agent, masking agent, coloring agent, flavoring agent, sweetening agent, or a combination thereof. The formulations may also include other active agents, for example, other therapeutic or prophylactic agents. As various combinations of agents are useful, the present compositions may also be described as specifically excluding any one or more the agents just described (for example, a given agent may exclude a surfactant). Pharmaceutically acceptable carriers can include any and all solvents, dispersion media, coatings, antibacterial and antifungal agents, isotonic and absorption delaying agents, and the like that are physiologically compatible, sterile aqueous solutions or dispersions and sterile powders for the extemporaneous preparation of sterile injectable solutions or dispersion. The use of such media and agents for pharmaceutically active substances is well known in the art. Supplementary active compounds can also be incorporated into the compositions.

Methods of making a pharmaceutical composition include admixing at least one active extract, fraction, or compound, as described herein, together with one or more other pharmaceutically acceptable ingredients, such as carriers, diluents, excipients, and the like. When formulated as discrete units, such as tablets or capsules, each unit contains a predetermined amount of the active compound. The formulations may be prepared by any methods well known in the art of pharmacy. The formulation may be prepared to provide for rapid or slow release; immediate, delayed, timed, or sustained release; or a combination thereof. Formulations may be in the form of liquids, solutions, suspensions, emulsions, elixirs, syrups, electuaries, mouthwashes, drops, tablets, granules, powders, lozenges, pastilles, capsules, gels, pastes, ointments, creams, lotions, oils, foams, sprays, mists, or aerosols. Formulations may be provided as a patch, adhesive plaster, bandage, dressing, or in the form of depot or reservoir. Many methods for the preparation of such formulations are known to those skilled in the art.

Routes of Administration:

The pharmaceutical and neutraceutical compositions of the present invention may be formulated for administration by any route of administration, including but not limited to systemic, peripheral, or topical. Illustrative routes of administration include, but are not limited to, oral, such as by ingestion, buccal, sublingual, transdermal including, such as by a patch, plaster, and the like, transmucosal including, such as by a patch, plaster, and the like, intranasal, such as by nasal spray, such as by inhalation or insufflation therapy using, such as via an aerosol through the mouth or nose, rectal, such as by suppository or enema, vaginal, such as by pessary, parenteral, such as by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and by implant of a depot or reservoir, such as intramuscularly. Methods of preparing pharmaceutical formulations are well known in the art. Dosage of the pharmaceutical compositions may vary by route of administration. Certain administration methods may include the step of administering the composition one or more times a day to obtain the desired therapeutic effect. In certain further embodiments, modes of administration can include tablets, pills, and capsules, all of which can be formulated by one of ordinary skill in the art.

### EXAMPLES

### Example 1

### Fractionation of SPGE

Because SPGE is non-toxic and has been shown to inhibit prostate cancer growth both in vitro and in vivo (Karna et al.), we wanted to investigate the nature of the compounds present in the whole extract. To this end, we employed a “top-down” logic wherein we fractionated the whole extract using classical column chromatography followed by sequential separation of sub-fractions from the complex whole extract based upon physicochemical characteristics such as polarity and solubility. We then performed a comparative quantitation of total polyphenolic content of all 7 SPGE fractions.

Sweet potato greens extract preparation: We obtained the Young Whatley/Loretan (TU-155) variety of sweet potato (Ipomoea batatas) greens, harvested on day 30, and began preparing an extract by soaking air-dried leaves in methanol overnight for three consecutive days. The supernatant was collected daily and was finally concentrated in vacuo (Buchi Rotavap) followed by freeze-drying using a lyophilizer to a solid-powder form, which was stored at −80° C. until tested. We prepared a stock solution of SPGE by dissolving 10 mg of the extract in 1 ml of DMSO, and various concentrations were obtained by appropriate dilutions. Batch-to-batch variation was evaluated by analysis of polyphenolic content in SPGE by the Folin-Ciocalteu (FC) method.

Fractionation of SPGE:

To achieve optimal fractionation of SPGE, we employed a mobile phase system that ranged from the non-polar hexanes to highly polar methanol. For this, classical column chromatographic separation was performed on SPGE (3 g) that was loaded on to a silica gel column, which was run down using a hexane-ethyl acetate solvent system starting with 500 ml of 100% hexane. The fraction was collected in a conical flask and stored at 4° C. This was followed by elution using 500 ml of hexane:ethyl acetate solution (90:10). Subsequently, a gradient increase in the percentage of ethyl acetate (10% each time) was incurred in the mobile phase to elute various components of SPGE into different fractions. After the elution of 50:50 hexane:ethyl acetate fraction, hexane was replaced by 50% methanol to elute the highly polar components. With an increment of 10% methanol each time (starting from 50:50 methanol:ethyl acetate) the column was finally eluted with 100% methanol to ensure complete elution of all components. A total of 17 fractions thus obtained were concentrated in vacuo (Buchi Rotavap) followed by separation on thin layer chromatography (TLC). Based on the observed bands, fractions with similar TLC profiles (Rf values) were pooled to finally obtain 7 fractions (F1-F7). All 7 fractions were freeze-dried using a lyophilizer and were stored at −80° C. until tested.

Determination of Total Phenol Content:

Total phenolic content was determined by the FC method using chlorogenic acid as the standard. Chlorogenic acid (0.5 g) was dissolved in 10 ml ethanol and then diluted to 100 ml with water to make a final concentration of 5 g/L. 50, 100, 250, and 500 mg/L concentrations of standards and 0.5, 1, 2, 3, 4 and 5 mg/ml concentrations of test extracts were prepared in distilled water. Twenty microliters of standard or test extract was dissolved in 1.58 ml water, followed by 100 μl FC reagent. This mixture was mixed thoroughly and incubated no longer than 8 minutes. Sodium carbonate solution (300 μl) was added to the above mixture and was incubated for 2 hours at room temperature. A final volume of 2 ml was measured for absorbance at 765 nm and the results were expressed as milligrams of chlorogenic acid equivalents (ChAE) per gram dry material (ChAE mg/g). The linear range of the calibration curve was 0.02 to 0.2 mg/ml. All samples were analyzed in triplicate.

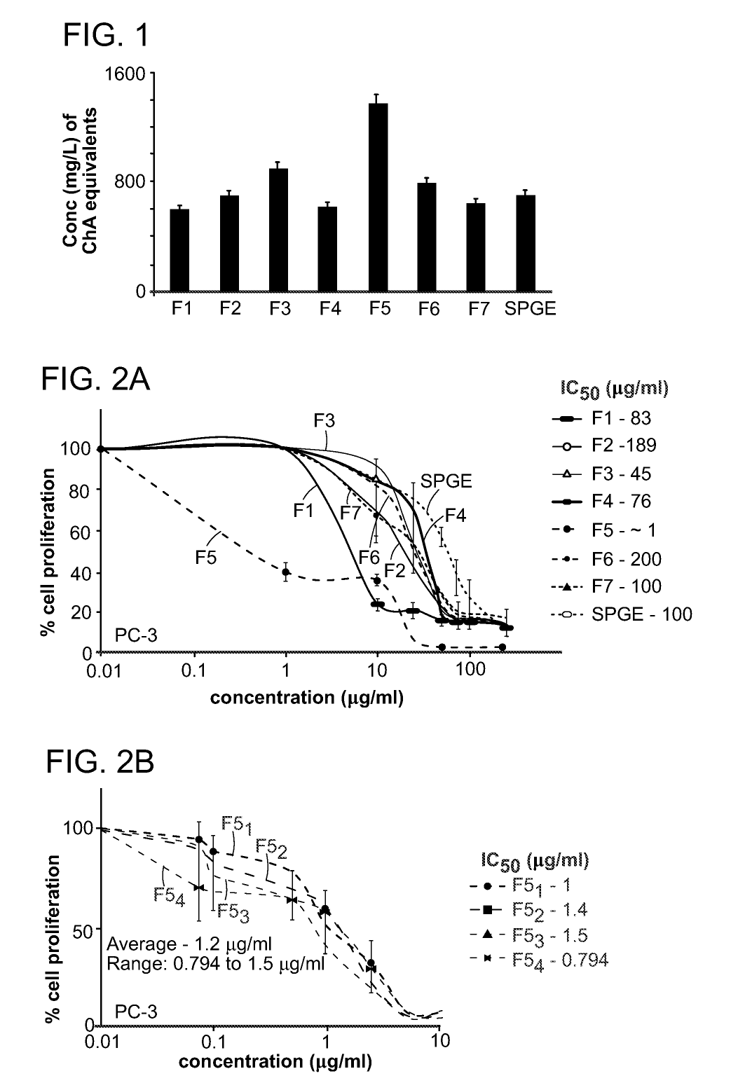

The quantitative comparison of all 7 SPGE fractions revealed that F5 contains ˜2-fold higher phenolic content as compared to SPGE (FIG. 1). Given these data, together with the observation that polyphenolic content is correlated with bioactivity, we next examined the in vitro efficacy of the various SPGE fractions.

Statistical analysis was carried out for all experiments. Briefly, the mean and standard deviations were calculated for all quantitative experiments using Microsoft-Excel software. The Student's t-test was used to determine the differences between groups with p-values of ≦0.05 considered as statistically significant.

### Example 2

### F5 Exhibits Antiproliferative Activity in Prostrate Cells

To determine the half-maximal concentration of growth inhibition (IC₅₀) for the 7 SPGE fractions in PC-3 cells, we carried out the MTT assay, and to evaluate the capacity of a cell to proliferate to form a colony upon removal of the drug, we used a colony survival assay.

Cell Culture and Materials:

Human prostate cancer cells (PC-3 cells) were cultured in RPMI-1640 media (Mediatech, Inc., Manassas, Va.) combined with 10% heat-inactivated fetal bovine serum (FBS; Hyclone, Logan, Utah) and 1% penicillin/streptomycin solution. Cells were cultured in a humidified atmosphere at 37° C. and 5% CO₂. MTT dye (thiazolyl blue tetrazolium bromide, 98% TLC), dimethyl sulfoxide (DMSO), QA, ChA, CA, FC reagent, ACS grade methanol, ethyl acetate, hexanes and high-performance liquid chromatography (HPLC) grade solvents were from Sigma-Aldrich (St. Louis, Mo.). Stably-transfected luciferase-expressing PC-3 cells (PC-3-luc cells) and luciferin were from Caliper Life Sciences (Alameda, Calif.).

In Vitro Proliferation Assay:

Briefly, 5×10³ cells/well in a 96-well format were treated with gradient concentrations of test fractions dissolved in DMSO (0.1%). The concentrations used were 1, 10, 25, 50, 75, 100 and 250 μg/ml. F5 was further tested at lower concentrations (0.075, 0.1, 0.5, 1, 5 and 10 μg/ml). After a 48-hour incubation, cells were washed with PBS followed by addition of 5 mg/ml MTT solution. Cells were then incubated at 37° C. in the dark for 4 hours. The formazan product was dissolved by adding 100 μl of 100% DMSO after removing the medium from each well. The absorbance was measured at 570 nm using a Spectra Max Plus multi-well plate reader (Molecular Devices, Sunnyvale, Calif.).

Colony Survival Assay:

PC-3 cells (1000) were seeded in a 6-well plate and were treated with 10 μg/ml F5 for 24 and 36 hours, then washed, and cultured with regular RPMI medium (including the controls). After 7 days, each well was washed with PBS, fixed, stained with the clonogenic reagent for 20 minutes, and then rinsed with tap water. The stained colonies (control and treated) were then counted. A colony was arbitrarily defined to consist of at least 50 cells.

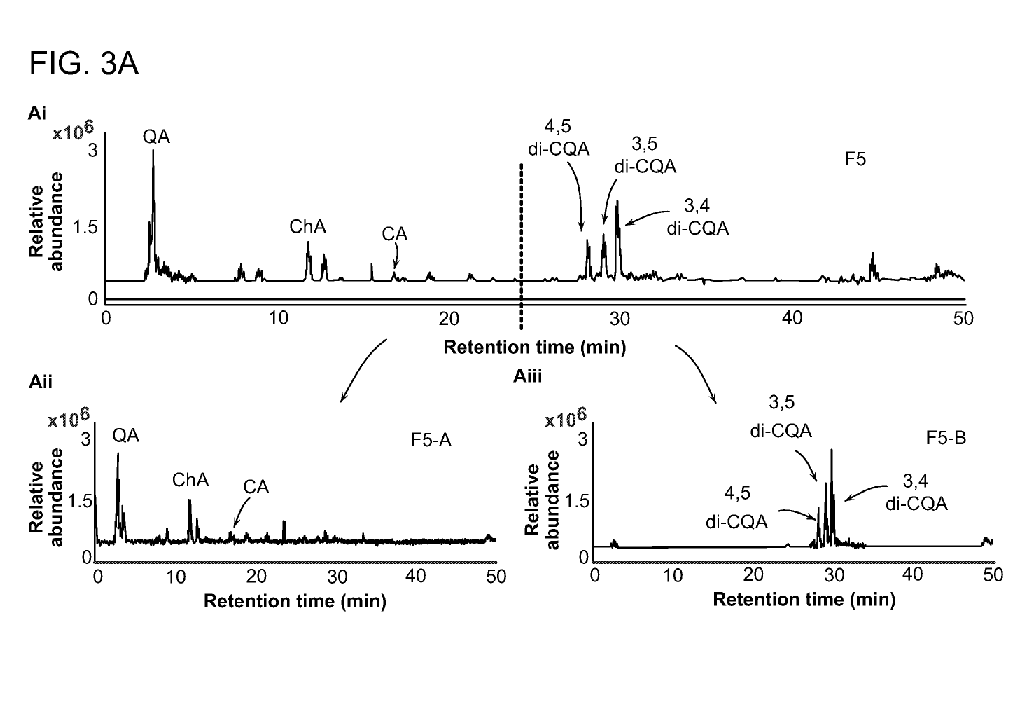

The IC₅₀ values of F1-F7 were in the range of ˜1-200 μg/ml (FIG. 2A). Indeed, the differential total phenolic content and polarity of various components that define a fraction might underlie the range of antiproliferative activity displayed by these fractions. Fraction 5 was the most active among the 7 fractions. Its IC₅₀ value was initially calculated to be approximately 1 μg/ml (FIG. 2A). To precisely determine the IC₅₀ value of F5, we then tested its efficacy at lower concentrations of F5 subfraction (0.075, 0.1, 0.5, 1, 5 and 10 μg/ml) obtained from 4 different batches (F5₁, F5₂, F5₃ and F5₄) in PC-3 cells (FIG. 2B). The IC₅₀ of F5 was found to be within a range of 0.794-1.5 μg/ml (FIG. 2B), which was ˜100 fold more potent compared to the whole SPGE extract (IC₅₀=100 μg/ml). In addition, F5 exhibited better efficacy in other prostate cancer cell lines (LNCaP, 22Rv1, DU145 and C4-2) compared to SPGE, suggesting the effect of fraction 5 on cell survival is generally applicable to a variety of prostate cancer cells.

We also carried out a colony formation assay to evaluate the capacity of a cell to proliferate to form a colony after removal of F5. Fraction 5 demonstrated significant anti-proliferative activity at the two time points (24 h and 26 h) evaluated compared to control.

### Example 3

### F5 Shows Enrichment of Major Phenolic Components of SPGE

Having shown the differential bioactivity of SPGE fractions, we carried out a comparative quantitation of the phenolics present in the 7 SPGE fractions by LC-UV/MS analysis.

HPLC with UV and Mass-Spectrometric Detection:

HPLC-UV separation of the 7 fractions was carried out on a HP1100 series Instrument (Agilent Technologies, Wilmington, Del.) equipped with a photodiode array detector, using an Agilent Zorbax reversed phase (SB-C18, 3.0×250 mm, 5.0 μm) column. The mobile phase system consisting of solvent A (0.1% formic acid in water) and solvent B (ACN) was used to achieve the separations. The gradient elution was set as follows: starting at 10% B, achieving 20% B at 20 minutes followed by 60% B over the next 20 minutes, which was held for an additional 10 minutes; reconditioning to 10% B at 51 minutes and ending the run at 60 minutes with a flow rate of 0.4 ml/minutes. Ten microliters of each fraction (1.0 mg/ml), dissolved and filtered in 25% ACN, was injected into the system and the resultant HPLC-UV peaks were detected at 326 nm.

The HPLC-MS analyses were performed in tandem with HPLC-UV using the Agilent Zorbax reversed phase (SB-C18, 3.0×250 mm, 5.0 μm) column interfaced to an Agilent 6400 series Triple quadrupole LC/MS equipped with an electrospray ionization source, operated in negative-ion mode. The nebulizer and collision gases were nitrogen and helium, respectively, with the former set at 40 psi. A drying gas temperature of 300° C., drying gas flow rate of 9 L/min and capillary voltage of −3000V were the spray chamber parameters. The presence of quinic (QA, m/z=191, RT: 2.7 min), chlorogenic (ChA, m/z=353, RT: 11.6 min) and caffeic (CA, m/z=179, RT: 15.5 min) acids in the fractions was confirmed using selected ion monitoring (SIM) and the HPLC retention time (RT) of the same in all the fractions against pure standards.

Three major phenolics, quinic (QA, m/z=191), caffeic (CA, m/z=179) and chlorogenic (ChA, m/z=353) acids were identified as present in SPGE (Karna et al.), along with other isochlorogenic acids like 4,5-di-caffeoylquinic acid (4,5-di-CQA, m/z=515), 3.5-di-caffeoylquinic acid (3,5-di-CQA, m/z=515) and 3,4-di-caffeoylquinic acid (3,4-di-CQA, m/z=515). The results of further analysis of F1 through F7 compared to SPGE demonstrated a relative abundance of the major phenols, QA, CA and ChA (Table 1; values representing the average of three independent experiments). The concentrations of QA, CA and ChA were quantified using pure standards. For example, F1 and F2, the fractions of lower polarity, showed an absence of QA, CA and 4,5-di-CQA. The CA content was found to be high in F3 and F4 as opposed to ChA (Table 1). The most active fraction, however, F5 exhibited the highest amounts of QA and ChA. The enrichment of 4,5-di-CQA, 3,4-di-CQA and 3,4-di-CQA was observed from F3 onwards.

TABLE 1

| Sample | Concentration (μg/mg) | | |
|---|---|---|---|
| Name | Quinic acid | Chlorogenic acid | Caffeic acid |
| F1 | — | 0.39 | — |
| F2 | 1.60 | 0.38 | — |
| F3 | 2.55 | 0.77 | 3.99 |
| F4 | 1.27 | 1.62 | 1.98 |
| F5 | 115.09 | 18.94 | 0.13 |
| F6 | 55.81 | 6.28 | 0.14 |
| F7 | 27.6 | 6.70 | 0.13 |
| SPGE | 44.98 | 5.29 | 2.61 |

The concentrations of isochlorogenic acids were also quantified using pure standards. The results are shown in Table 2 and represent the average of three independent experiments. We observed decreased quantities in F6 and F7 as compared to F4 and F5. F4 was found to be enriched in all three isochlorogenic acids with 3,5-di-CQA being the most abundant, whereas the content of 3,4-di-CQA is enhanced in F5. Fractions 6 and 7 exhibited a decrease in the composition of isochlorogenic acids. These data confirmed the differential abundance of several phenolic compounds in F5. Using the same methods described above, different amounts of bioactive constituents like neochlorogenic acid (nChA, m/z=353), cryptochlorogenic acid (cChA, m/z=353), quercetin-glucoside (QnG, m/z=463) and its isomer (QnG isomer, m/z=463), quercetin (Qn, m/z=447) and astragalin (AGN, m/z=447) were identified in SPGE and compared to F5.

TABLE 2

| | Quantitation (area under the curve) | | |
|---|---|---|---|
| | 4,5-di-O- | 3,5-di-O | 3,4-di-O |
| Sample | caffeoylquinic acid | caffeoylquinic acid | caffeoylquinic acid |
| F1 | — | 2.46 | 1.58 |
| F2 | 1.22 | — | 1.25 |
| F3 | 20.72 | 2.95 | 7.57 |
| F4 | 250.88 | 741.96 | 228.82 |
| F5 | 144.53 | 424.18 | 669.04 |
| F6 | 64.86 | 189.17 | 191.92 |
| F7 | 116.34 | 258.32 | 245.59 |
| SPGE | 166.25 | 483.23 | 228.45 |

Next, the hydroxycinnamic acids, ChA and CA along with QA in SPGE and F5 were quantitated using the respective pure standards. Tandem-mass spectrometric analysis affirmed the presence of QA, ChA and CA in SPGE and F5. The selected ion monitoring (SIM) of QA (191), ChA (353) and CA (179) confirmed their elution in both F5 and SPGE exactly at the same retention times. Notably, the ratio between QA, ChA and CA differed between F5 and SPGE. The ratio of QA:ChA:CA in F5 has been calculated to be 6:1:0.005, whereas these same compounds existed in a 9:1:0.6 ratio in SPGE. On the other hand, the isochlorogenic acids were found to be in a ratio of 1:3:4.6 in F5 as compared to the 1:3:1.4 seen in SPGE. Furthermore, the chemical fingerprints of F5 and SPGE establish an obvious difference between their compositions. For example, the compound u4 with m/z value of 385 could not be observed in SPGE, whereas it was present at quantifiable levels in F5. QnG including 4 other unknown compounds, u5-u8, were absent in F5. Among the major phenolics, there was an approximately 2.6-(QA), 3.6-(ChA), and 3-(3,4-di-CQA) fold increase in F5 compared to SPGE. It is thus reasonable to speculate that these differences might be responsible for the higher bioactivity of F5 compared to SPGE in prostate cancer cells.

### Example 4

### Sub Fractionation of F5 Results in Loss of Bioactivity

Next, we examined whether sub-fractionating F5 into its constituent components could identify single agents that were more active compared to the whole fractions. Analytical liquid chromatography was used for this analysis.

Sub Fractionation of F5 Using Analytical HPLC-UV Chromatography:

The pure standards, QA, ChA and CA, were combined as a mixture to mimic their respective concentrations as quantitated in F5. This mixture was used at various increasing gradient concentrations to test its in vitro efficacy against PC-3 cells. Specifically, we used the concentrations 0.075, 0.1, 0.5, 1, 5, and 10 μg/ml. Cell proliferation was measured by MTT assay.

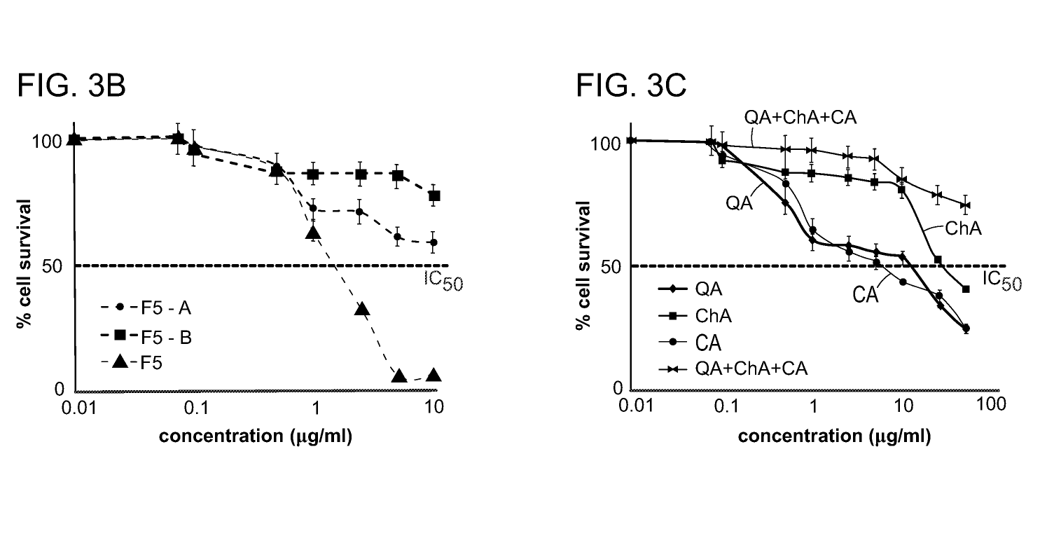

For these experiments, repeated injections of 10 μl of F5 were made into the HPLC system and the eluate from 0-25 minutes was collected as the sub-fraction F5-A. The remaining part, obtained from 25.1-50 minutes, was collected as sub-fraction F5-B (FIG. 3). F5-A is a combination of QA, ChA and CA, whereas F5-B constituted the 3 isochlorogenic acids. The sub-fractions thus obtained were concentrated and lyophilized. Next, we again employed a bioactivity-guided approach to determine the efficacy of F5 sub-fractions. Both the sub-fractions were reconstituted in DMSO to yield 1 mg/ml stock solutions which were then used to dose PC-3 cells at gradient concentrations for 48 hours. An MTT assay performed post-incubation showed that neither of the individual sub-fractions was as active as F5. Surprisingly, F5-A and F5-B did not show 50% inhibition of cell growth even at the highest test concentration (250 μg/ml) and hence, their IC₅₀ values could not be determined. This clearly indicated that F5-A needs F5-B and vice versa to mimic F5's activity, thus, suggesting a synergistic interplay among F5's constituents. Additionally, the clear differences between the compositions of each sub-fraction compelled us to investigate if the loss of activity in the sub-fractions was related to their respective compositions.

### Example 5

### F5 Phytochemicals Exhibit Synergism

To corroborate this observation, we next tested commercially-available QA, ChA and CA in combination at varying concentrations against PC-3 cells. Quantitative data points out that 1 mg of F5 contains 115 μg of QA, 16 μg of ChA and 0.1 μg of CA. Given that the IC₅₀ value of F5 is approximately 1 μg/ml (based on the range of 0.794-1.5 μg/ml), F5 (1 μg) actually consists of 115 ng QA, 16 ng ChA and 0.1 ng CA. Assuming that these three compounds are the major players that contribute to F5-A's activity, we tested the bioactivity of a mixture of the three pure standards by measuring the percentage of cell proliferation using the MTT assay (as described in Example 2). PC-3 cells were treated with this mixture in an increasing gradient concentration (0.075, 0.1, 0.5, 1, 5 and 10 μg/ml) ensuring that the relative quantities of the three compounds (QA+ChA+CA, the major constituents of F5-A) at each test concentration bore the same ratiometric relationship as was observed between them in F5. This mixture formulation thus mimicked the composition of F5-A (as it exists in F5). Evaluation of the in vitro efficacy of this subfraction might also enable exclusion of the possible antagonism of other yet unknown phytochemicals in F5-A. Our data suggests that even at the highest concentration tested (10 μg/ml), the formulated mixture of pure standards did not show 50% inhibition in cell growth. As the pure standard mixture of three compounds could not reproduce an efficacy equivalent to F5's efficacy, we speculate that the other unknown components in F5-A perhaps did not exert an antagonistic influence. Thus our results from in vitro experiments testing various combinations of pure standards (QA, ChA and CA) suggests that the higher efficacy of F5 could be ascribed not only to an enhanced total polyphenolic content, but also to possible synergistic interactions associated with definitive ratiometric composition of these phenolics.

The other sub-fraction, F5-B, was also tested and found to be non-active. Hence, the loss of bioactivity in both sub-fractions F5-A and F5-B individually suggests the existence of a synergism among the characterized and the yet unknown F5 components. It is perhaps likely that other identified compounds such as Qn, nChA, cChA, QnG, AGN contribute to uphold the superior activity of F5. These data also emphasize the importance of the occurrence of QA, ChA, CA, 4,5-diCQA, 3,5-di-CQA and 3,4-di-CQA in a distinct ratio, as found in F5 to display remarkable activity.

### Example 6

### Oral Feeding of F5 Inhibits Prostate Tumor Growth In Vivo

Given the significant difference in the in vitro anti-proliferative activity of F5 compared to SPGE, we evaluated the ability of F5 to inhibit human prostate tumor xenografts subcutaneously implanted in athymic nude mice. We used a PC-3 cell-line stably-expressing luciferase (PC-3-luc), which enables real-time visualization and longitudinal monitoring of prostate cancer growth non-invasively in mice.

In Vivo Tumor Growth and Bioluminescent Imaging:

PC-3-luc cells (1×10⁶) were subcutaneously injected in the right flank of six-week old male BALB/c nude mice (Harlan Laboratories, Inc., Indianapolis, Ind.). When mice developed palpable tumors, they were randomly divided into three groups of eight mice each. The control group received vehicle (PBS with 0.05% Tween-80, pH=7.4) and the treatment group received 400 mg/kg body weight F5 by oral gavage daily. Real-time bioluminescent imaging of luciferase activity in live mice was employed to monitor tumor growth using the IVIS in vivo imaging system (Caliper Life Sciences, Alameda, Calif.) with the Live Imaging software. Briefly, mice anesthesized with isoflurane were intraperitoneally injected 25 mg/ml luciferin and imaged with a CCD camera. An integration of 20 s with 4 binnings of 100 pixels was used for image acquisition. The relative photon count at the tumor site of the mice from vehicle or F5-treated groups was quantitated as the number of photons leaving a square cm of tissue and radiating into a solid angle of 1 steradian (photons/sec/cm²/sr).

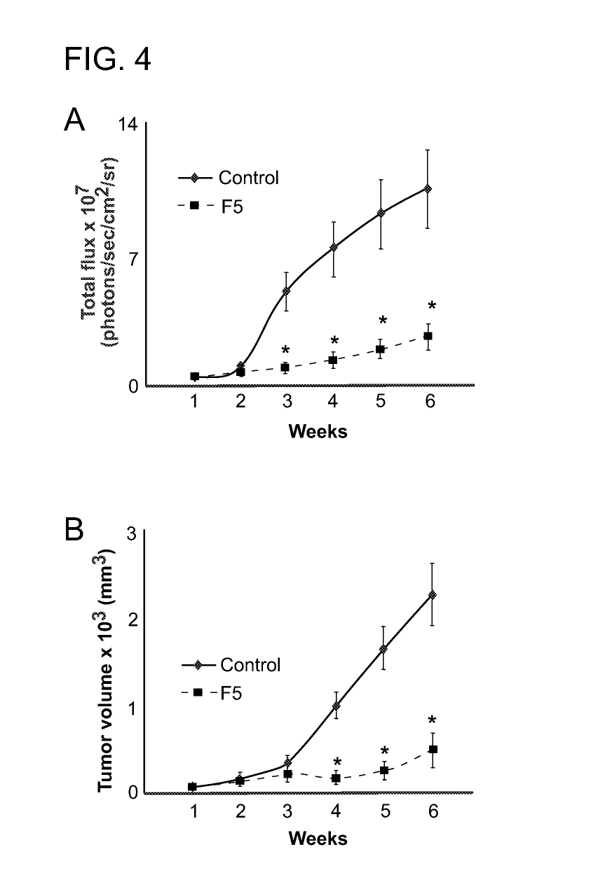

We have previously shown that SPGE inhibits the in vivo tumor growth by 69% (Karna et al.). We found that the treatment group fed with 400 mg/kg bw F5 daily by oral-gavage for six weeks showed a time-dependent inhibition of tumor growth (FIG. 4B), compared to vehicle-treated control animals. A relative total flux quantitation revealed a ˜75% inhibition in tumor-volume with a confidence level of p<0.05 (n=8, FIG. 4B) as measured at week 6 for the F5-fed group compared to vehicle-treated controls. Body weights were recorded twice a week to evaluate the general-health and well-being of animals during treatment. Mice in the F5 treatment group exhibited normal weight gain with no signs of discomfort during the treatment regimen. All animals in the control group were euthanized due to tumor overburden, in compliance with institutional IACUC guidelines. At the end of week 6, the excised tumors were weighed post-euthanasia and a ˜74% reduction in tumor weight was observed in F5-treated groups respectively, compared to controls.

### Example 7

### F5 Mediates Apoptosis and Reduction of Tumor Growth In Vivo

Immunoblot Analysis and Immunofluorescent Microscopy:

To evaluate in vivo inhibition of tumor growth upon oral feeding of F5, immunoblot and immunofluorescent microscopy techniques were used. Specifically, we immunostained for Ki67 (MIB-1), a well-known marker of cell proliferation; the Ki67 antigen is a non-histone protein expressed in all phases of the cell cycle except G0. First, tumor lysates treated with vehicle and 400 mg/kg bw F5 were subjected to western blot analysis. Membranes were then probed for cleaved caspase-3 and cleaved PARP along with β-actin, which was used as a loading control. Paraffin-embedded tumor sections from control and F5-treated groups were processed and immunostained with apoptotic markers, cleaved caspase-3 and cleaved PARP, and the proliferation marker, Ki67. Fluorescent images were captured using confocal microscopy. Next, human prostate cancer (PC-3) cells were treated with 10 μg/ml F5 and cell lysates were collected at 0, 6, 12, 24, and 48 h. Immunoblot analysis was performed on the F5-treated and control samples by probing for cleaved PARP and β-actin to confirm the induction of apoptosis.

Our results show that Ki67-stained tumor sections from F5-fed animals showed decreased immunoreactivity compared to vehicle-fed animals. Tumor sections from F5-treated groups also showed an increase in cleaved caspase-3 and PARP staining compared with vehicle-fed controls, suggesting induction of robust apoptosis in tumors from SPGE-treated mice.

Furthermore, the tumor tissue lysates were immunoblotted for cyclin D1, and the apoptotic markers, cleaved caspase-3 and cleaved PARP. Cyclin D1 plays a central role in the regulation of proliferation, linking extracellular signaling environment to cell cycle progression. There was a decrease in cyclin D1 expression in F5-fed tumor lysates suggesting a cessation of cell cycle progression. Further, as expected, the cleaved caspase-3 and PARP expression were higher in F5-treated tumors compared to controls. Similar trend was observed in PC-3 cell lysates, where F5 treatment showed increased cleaved PARP expression compared to controls.

### Example 8

### Histopathological Analysis

Toxicity is a concern and is often observed in prostate cancer patients undergoing either radio- or chemotherapy. To assess the degree of toxicity, a complete histopathological evaluation of the major organs was conducted. For this, mice were euthanized after 6 weeks of F5 or vehicle feeding by exposing to CO₂ for 2 min. Blood was collected by cardiocentesis in accordance with our standard IACUC protocol. The organs were immediately collected, formalin-fixed and paraffin-embedded. Sections (5 μm) were stained with hematoxylin and eosin (H&E). Microscopic evaluation was performed by a pathologist in a blinded manner.

The histopathological evaluation of the tissues of intestine, spleen, liver, lung, brain, heart, adrenal gland, and testes from both vehicle- and F5-fed mice, revealed no detectable differences in architecture. Furthermore, analysis of biochemical markers in the sera (alanine transaminase, aspartate transaminase, alkaline phosphate, lactic acid dehydrogenase, creatinine kinase, and urea nitrogen) collected from both vehicle- and F5-fed mice was observed to be within the normal range.

1. A method of identifying a fraction of a sweet potato greens extract (SPGE), the method comprising:
(a) providing a SPGE;
(b) fractionating the SPGE; and
(c) identifying the fraction or fractions that have (i) an improved ability to inhibit the proliferation of cancer cells relative to an unfractionated SPGE and (ii) a higher concentration of quinic acid and chlorogenic acid than an unfractionated SPGE.

2. The method of claim 1, further comprising:
(d) isolating the fraction or fractions identified in step (c) and, optionally, formulating the fraction or fractions in unit dosage form for oral administration.

3. The method of claim 1, wherein the SPGE is prepared from the sweet potato Ipomoea batatas.

4. The method of claim 1, wherein the SPGE is prepared by a method comprising the steps of soaking air-dried sweet potato leaves in an alcohol for about three consecutive days; collecting the supernatant; concentrating the supernatant in vacuo; and freeze-drying the supernatant to a solid-powder form.

5. The method of claim 1, wherein fractionating the SPGE comprises passing the SPGE over a silica gel column and eluting fractions of the SPGE from the column.

6. The method of claim 5, wherein eluting fractions of the SPGE from the column comprises elution with (a) 100% hexane; (b) a hexane- and ethyl acetate-containing solution, wherein the ratio of hexane to ethyl acetate changes from primarily hexane to primarily ethyl acetate over the course of subsequent elutions; (c) an ethyl acetate- and methanol-containing solution, wherein the ratio of methanol to ethyl acetate changes from primarily ethyl acetate to primarily methanol over the course of subsequent elutions; and (d) 100% methanol.

7. The method of claim 1, wherein fractionating the SPGE comprises column chromatography and thin layer chromatography (TLC).

8. The method of claim 7, wherein the fractions obtained following column chromatography are concentrated in vacuo and then characterized by TLC.

9. The method of claim 8, wherein the fractions with similar TLC profiles (R_{f} values) are pooled.

10. The method of claim 7, wherein fractionating the SPGE by column chromatography produces about 12-18 fractions and subsequent fractionation by TLC produces about 5-10 fractions.

11. The method of claim 10, wherein fractionating the SPGE by column chromatography produces 17 fractions and subsequent fractionation by TLC produces 7 fractions.

12. The method of claim 11, wherein a fraction obtained by the subsequent fractionation by TLC has (i) an improved ability to inhibit the proliferation of cancer cells relative to an unfractionated SPGE and (ii) a composition with a higher concentration of QA and ChA than an unfractionated SPGE.

13. The method of claim 12, wherein the fraction further comprises neochlorogenic acid, cryptochlorogenic acid, quercetin-glucoside, quercetin, or astragalin.

14. The method of claim 1, wherein the SPGE is obtained from extraction with carbon dioxide, extraction with a supercritical fluid, or distillation with water.

15. A physiologically acceptable formulation comprising a fraction of SPGE made by the method of claim 1 and optionally further comprising an inhibitor of UGT (a UDP-glucuronosyltransferase).

16. A physiologically acceptable formulation comprising a fraction of SPGE, wherein the fraction comprises quinic acid (QA), chlorogenic acid (ChA) and caffeic acid (CA) in amounts or in a ratio relative to one another that is different from that found in a comparable, unfractionated SPGE, wherein the formulation further, optionally, comprises an inhibitor of UGT (a UDP-glucuronosyltransferase).

17. The formulation of claim 16, wherein the fraction comprises elevated levels of quinic acid and/or chlorogenic acid relative to the levels found in a comparable, unfractionated SPGE.

18. The formulation of claim 17, wherein the fraction comprises at least 2.5 times the amount of quinic acid as in the comparable, unfractionated SPGE and/or at least 2.5 times the amount of chlorogenic acid in the comparable, unfractionated SPGE.

19. The formulation of claim 16, wherein the ratio of QA:ChA:CA is about 6 to 1 to 0.007.

20. The formulation of claim 16, wherein the ratio of ChA to CA is less than 1:1.

21. The formulation of claim 16, further comprising neochlorogenic acid, cryptochloro-genic acid, quercetin-glucoside, quercetin, or astragalin.

22. The formulation of claim 16, further comprising an excipient.

23. The formulation of claim 22, wherein the excipient is a filler, hydrogel, buffer, coloring agent, or flavoring agent.

24. The formulation of claim 22, wherein the formulation is suitable for oral or topical administration.

25. A method of reducing the risk that a subject will develop cancer, hypertension, or diabetes, the method comprising administering to the subject an effective amount of the physiologically acceptable formulation of claim 15.
